(19) United States
(12) Patent Application Publication
(10) Pub. No.: US 2010/0030130 A1
(43) Pub. Date: Feb. 4, 2010

(54) PHARMACEUTICAL INTERVENTION FOR MODULATION OF NEURAL PLASTICITY

(75) Inventors: John L. Parker, Roseville (AU); Dusan Milojevic, Wheelers Hill (AU); Claudia Tasche, Elanora Heights (AU)

Correspondence Address: CONNOLLY BOVE LODGE & HUTZ LLP, 1875 EYE STREET, N.W., SUITE 1100, WASHINGTON, DC, 20006, US

(73) Assignee: COCHLEAR LIMITED, Lane Cove (AU)

(21) Appl. No.: 12/415,812

(22) Filed: Mar. 31, 2009

Related U.S. Application Data
(63) Continuation-in-part of application No. 11/045,624, filed on Jan. 28, 2005.
Application No. 11/045,624 is a continuation-in-part of application No. 10/494,995, filed on Sep. 23, 2004.
(60) Provisional application No. 61/041,185, filed on Mar. 31, 2008.

(30) Foreign Application Priority Data
Nov. 9, 2001 (AU) AU PR 8792

(51) Int. Cl. A61F 11/04; A61N 1/36; A61M 37/00

(52) U.S. Cl. 604/21; 607/3; 604/20

(57) ABSTRACT

A stimulating medical device. The stimulating medical device comprises: a stimulating assembly implantable proximate to a recipient's neural system, the assembly having at least one agent delivery port and a plurality of electrical contacts; an electrical stimulation controller configured to generate electrical stimulation signals for application the neural system via one or more of the plurality of electrical contacts; a pharmaceutical agent source configured to provide a plasticity modulating agent to the at least one delivery port for application to the neural system; and a pharmaceutical agent controller configured to control one or more of the pharmaceutical agent source and the at least one delivery port to cause selective application of the plasticity modulating agent to the neural system to at least one of reinforce and activate one or more neural pathways within the system.

### CROSS-REFERENCE TO RELATED APPLICATIONS

The present application claims the benefit of U.S. Provisional Patent Application 61/041,185; filed Mar. 31, 2008. This application is a continuation-in-part of U.S. patent application Ser. No. 11/045,624, entitled “Stimulating Device,” filed Jan. 28, 2005, which is a continuation-in-part of U.S. patent application Ser. No. 10/494,995, entitled “Subthreshold Stimulation of a Cochlea,” filed Sep. 23, 2004, which is a national stage application of PCT/AU02/01537, filed Nov. 11, 2002, which claims priority to Australian Provisional Application No. AU PR 8792, filed Nov. 9, 2001, the entire contents and disclosures of which are hereby incorporated by reference herein.

### BACKGROUND

1. Field of the Invention

The present invention relates generally to a stimulating medical device and, more particularly, to pharmaceutical intervention for modulation of neural plasticity.

2. Related Art

Medical devices having one or more implantable components, generally referred to as implantable medical devices herein, have provided a wide range of therapeutic benefits to patients over recent decades. As such, the type of implantable devices and the range of functions performed thereby have increased over the years. Particular types of implantable medical devices, referred to as stimulating medical devices, are used to stimulate the nerve cells of the device recipient. A notable use for such stimulating medical devices is in recipients who suffer from various forms of hearing loss.

Hearing loss, which may be due to many different causes, is generally of two types, conductive and sensorineural. In some cases, a person suffers from hearing loss of both types. Conductive hearing loss occurs when the normal mechanical pathways for sound to reach the cochlea, and thus the sensory hair cells therein, are impeded, for example, by damage to the ossicles. Individuals who suffer from conductive hearing loss typically have some form of residual hearing because the hair cells in the cochlea are undamaged. As a result, individuals suffering from conductive hearing loss typically receive an acoustic hearing aid. Acoustic hearing aids stimulate an individual's cochlea by providing an amplified sound to the cochlea that causes mechanical motion of the cochlear fluid.

In many people who are profoundly deaf, however, the reason for their deafness is sensorineural hearing loss. Sensorineural hearing loss occurs when there is damage to the inner ear, or to the nerve pathways from the inner ear to the brain. As such, those suffering from some forms of sensorineural hearing loss are thus unable to derive suitable benefit from conventional acoustic hearing aids. As a result, hearing prostheses that apply electrical stimulation signals to nerve cells of the recipient's auditory system have been developed to provide the sensations of hearing to persons whom do not derive adequate benefit from conventional hearing aids. Such electrically-stimulating hearing prostheses apply electrical stimulation to nerve cells of the recipient's auditory system thereby providing the recipient with a hearing percept.

As used herein, the recipient's auditory system includes all sensory system components used to perceive a sound signal, such as hearing sensation receptors, neural pathways, including the auditory nerve and spiral ganglion cells, and parts of the brain used to sense sounds. Hearing prostheses that apply electrical stimulation signals to the recipient include, for example, auditory brain stimulators and cochlear™ prostheses (commonly referred to as cochlear™ prosthetic devices, cochlear™ implants, cochlear™ devices, and the like; simply “cochlear implants” herein.)

Oftentimes sensorineural hearing loss is due to the absence or destruction of the cochlear hair cells which transduce acoustic signals into nerve impulses. It is for this purpose that cochlear implants have been developed. Conventional cochlear implants provide a recipient with a hearing percept by delivering electrical stimulation signals directly to the auditory nerve cells, thereby bypassing absent or defective hair cells that normally transduce acoustic vibrations into neural activity. Such devices generally use an electrode array implanted in the cochlea so that the electrodes may differentially activate auditory neurons that normally encode differential pitches of sound.

### SUMMARY

In one aspect of the present invention, a cochlear implant is provided. The cochlear implant comprises: a stimulating assembly implantable in a cochlea of a recipient having at least one agent delivery port and a plurality of electrical contacts; an electrical stimulation controller configured to generate electrical stimulation signals for application to a first population of cochlea nerve cells via one or more of the plurality of electrical contacts; a pharmaceutical agent source configured to provide a plasticity modulating agent to the at least one delivery port for application to a second population of cochlea nerve cells; and a pharmaceutical agent controller configured to control one or more of the pharmaceutical agent source and the at least one delivery port to cause selective application of the plasticity modulating agent to the second population of nerve cells to at least one of reinforce and activate one or more neural pathways within the recipient's central auditory system.

In another aspect of the present invention, a method for modulating the neural plasticity of a recipient's auditory system with an implant comprising a stimulating assembly implantable proximate to the auditory system, the assembly having at least one agent delivery port and a plurality of electrical contacts. The method comprises: generating electrical stimulation signals; applying the electrical stimulation signals to a first population of cochlea nerve cells via one or more of the plurality of electrical contacts; and applying a plasticity modulating agent to a second population of cochlea nerve cells via the at least one delivery port to at least one of reinforce and activate one or more neural pathways within the recipient's central auditory system.

In a still other aspect of the present invention, a stimulating medical device is provided. The stimulating medical device comprises: a stimulating assembly implantable proximate to a recipient's neural system, the assembly having at least one agent delivery port and a plurality of electrical contacts; an electrical stimulation controller configured to generate electrical stimulation signals for application the neural system via one or more of the plurality of electrical contacts; a pharmaceutical agent source configured to provide a plasticity modulating agent to the at least one delivery port for application to the neural system; and a pharmaceutical agent controller configured to control one or more of the pharmaceutical agent source and the at least one delivery port to cause selective application of the plasticity modulating agent to the neural system to at least one of reinforce and activate one or more neural pathways within the system.

### BRIEF DESCRIPTION OF THE DRAWINGS

Embodiments of the present invention are described below with reference to the attached drawings, in which:

FIG. 1 is a perspective view of an implanted cochlear implant which may be advantageously configured to implement embodiments of the present invention;

FIG. 2A is a perspective, partially cut-away view of a cochlea exposing the canals and nerve fibers of the cochlea;

FIG. 2B is a cross-sectional view of one turn of the canals of a human cochlea;

FIG. 3A is graph illustrating the various phases of an idealized action potential as the potential passes through a nerve cell, illustrated in membrane voltage versus time;

FIG. 3B is a schematic diagram of the human central auditory system;

FIG. 4 is a detailed functional block diagram illustrating the components of a cochlear implant in accordance with embodiments of the present invention;

FIG. 5 is a side view of the implantable component of a cochlear implant in accordance with embodiments of the present invention;

FIG. 6 is a cross-sectional side view of a portion of a stimulating assembly in accordance with embodiments of the present invention;

FIG. 7A is a side view of an implantable stimulating assembly in accordance with embodiments of the present invention;

FIG. 7B is a side view of an implantable stimulating assembly in accordance with embodiments of the present invention;

FIG. 8 is a diagram of a pharmaceutical agent source in accordance with embodiments of the present invention;

FIG. 9 is a diagram of a pharmaceutical agent source in accordance with embodiments of the present invention;

FIG. 10 is a flowchart illustrating the operations performed by a cochlear implant in accordance with embodiments of the present invention;

FIG. 11 is a detailed flowchart illustrating the operations performed by a cochlear implant in accordance with embodiments of FIG. 10; and

FIG. 12 is a detailed flowchart illustrating the operations performed by a cochlear implant in accordance with embodiments of FIG. 10.

### DETAILED DESCRIPTION

Aspects of the present invention are generally directed to a stimulating medical device configured to apply a neural plasticity modulating agent to the neural system of a recipient. The plasticity modulating agent influences and/or controls the neural plasticity of the system.

Specifically, the stimulating medical device comprises an implantable stimulating assembly having delivery ports and electrical contacts. A pharmaceutical agent source delivers the plasticity modulating agent to the recipient's nerve cells via the delivery ports, and an electrical stimulation controller generates electrical stimulation signals that are applied to the nerve cells via the electrical contacts.

Embodiments of the present invention may be implemented in various types of stimulating medical devices such as functional electrical stimulators, cochlear™ prostheses (commonly referred to as cochlear™ prosthetic devices, cochlear™ implants, cochlear™ devices, and the like; simply “cochlear implants” herein), auditory brain stimulators, etc. As noted, cochlear implants stimulate auditory nerve cells, bypassing absent or defective hair cells that normally transduce acoustic vibrations into neural activity. Conventional cochlear implants generally use an array of electrodes, sometimes referred to as electrical contacts herein, inserted into or adjacent the cochlea so that the electrical contacts may activate auditory neurons that normally encode differential pitches of sound. Auditory brain stimulators are used to treat a smaller number of recipients, such as those with bilateral degeneration of the auditory nerve. The auditory brain stimulator comprises an array of electrical contacts configured to be positioned, for example, proximal to the recipient's brainstem. When implanted, the electrical contacts apply electrical stimulation signals to the cochlear nucleus in the brainstem, resulting in a hearing sensation by the recipient. For ease of illustration, the present invention will be described herein primarily in connection with cochlear implants. However, it should be appreciated that embodiments of the present invention, regardless of whether described herein, may be implemented in any stimulating medical device now known or later developed.

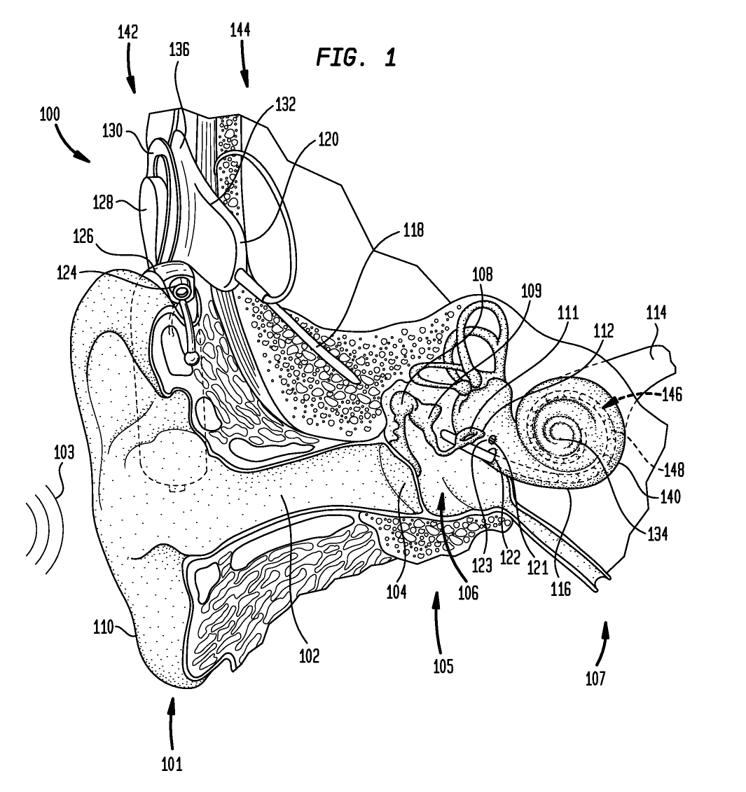

FIG. 1 is a perspective view of an exemplary cochlear implant 120 in which embodiments of the present invention may be implemented. The relevant components of the recipient's outer, middle and inner ear are described below, followed by a description of cochlear implant 120.

In a fully functional ear, outer ear 101 comprises an auricle 110 and an ear canal 102. An acoustic pressure or sound wave 103 is collected by auricle 110 and channeled into and through ear canal 102. Disposed across the distal end of ear cannel 102 is a tympanic membrane 104 which vibrates in response to sound wave 103. This vibration is coupled to oval window or fenestra ovalis 112 through three bones of middle ear 105, collectively referred to as the ossicles 106 and comprising the malleus 108, the incus 109 and the stapes 111. Bones 108, 109 and 111 of middle ear 105 serve to filter and amplify sound wave 103, causing oval window 112 to articulate, or vibrate in response to vibration of tympanic membrane 104. This vibration sets up waves of fluid motion of the perilymph within cochlea 140. Such fluid motion, in turn, activates tiny hair cells (not shown) inside cochlea 140. Activation of the hair cells causes appropriate nerve impulses to be generated and transferred via neural pathways through the spiral ganglion cells (not shown) and auditory nerve 114 to the brain (also not shown) where they are perceived as sound.

Cochlear implant 100 comprises an external component 142 which is directly or indirectly attached to the body of the recipient, and an internal component 144 which is temporarily or permanently implanted in the recipient. External component 142 typically comprises one or more acoustic pickup devices, such as microphone 124, for detecting sound, a sound processing unit 126, a power source (not shown), and an external transmitter unit 128. External transmitter unit 128 comprises an external coil 130 and, preferably, a magnet (not shown) secured directly or indirectly to external coil 130. Sound processing unit 126 processes the output of a sound input component, shown as microphone 124, that is positioned in the depicted embodiment adjacent auricle 110 of the recipient. Sound processing unit 126 generates encoded signals, sometimes referred to herein as encoded data signals, which are provided to external transmitter unit 128 via a cable (not shown).

Internal component 144 comprises, in this depicted embodiment, an internal receiver unit 132, a stimulator unit 120, and an elongate stimulating assembly 118. Internal receiver unit 132 comprises an internal coil 136, and preferably, a magnet (also not shown) fixed relative to the internal coil. Internal receiver unit 132 and stimulator unit 120 are hermetically sealed within a biocompatible housing, sometimes collectively referred to as a stimulator/receiver unit. The magnets facilitate the operational alignment of the external and internal coils, enabling internal coil 136 to receive power and stimulation data from external coil 130, as noted above. Elongate stimulating assembly 118 has a proximal end connected to stimulator unit 120, and a distal end implanted in cochlea 140. Electrode assembly 118 extends from stimulator unit 120 to cochlea 140 through mastoid bone 119. In some embodiments, stimulating assembly 118 may be implanted at least in basal region 116, and sometimes further. For example, stimulating assembly 118 may extend towards apical end of cochlea 140, referred to as cochlea apex 134. In certain circumstances, stimulating assembly 118 may be inserted into cochlea 140 via a cochleostomy 122. In other circumstances, a cochleostomy may be formed through round window 121, oval window 112, the promontory 123 or through an apical turn 147 of cochlea 140.

Stimulating assembly 118 comprises a longitudinally aligned and distally extending array 146 of stimulating electrical contacts 148, sometimes referred to as contact array 146 herein, disposed along a length thereof Although contact array 146 may be disposed on stimulating assembly 118, in most practical applications, contact array 146 is integrated into stimulating assembly 118. As such, for all embodiments of stimulating assembly 118, contact array 146 is generally referred to herein as being disposed in stimulating assembly 118. As described below, stimulator unit 120 generates stimulation signals which are applied by contacts 148 to cochlea 140, thereby stimulating auditory nerve 114.

In certain embodiments, external coil 130 transmits electrical signals (i.e., power and stimulation data) to internal coil 136 via a radio frequency (RF) link, as noted above. Internal coil 136 is typically a wire antenna coil comprised of multiple turns of electrically insulated single-strand or multi-strand platinum or gold wire. The electrical insulation of internal coil 136 is provided by a flexible silicone molding (not shown). In use, implantable receiver unit 132 maybe positioned in a recess of the temporal bone adjacent auricle 110 of the recipient.

Although FIG. 1 illustrates a cochlear implant 100 having an external component 142, it should be appreciated that embodiments of the present invention may be implemented in other cochlear implant embodiments, such as a totally implantable cochlear implant.

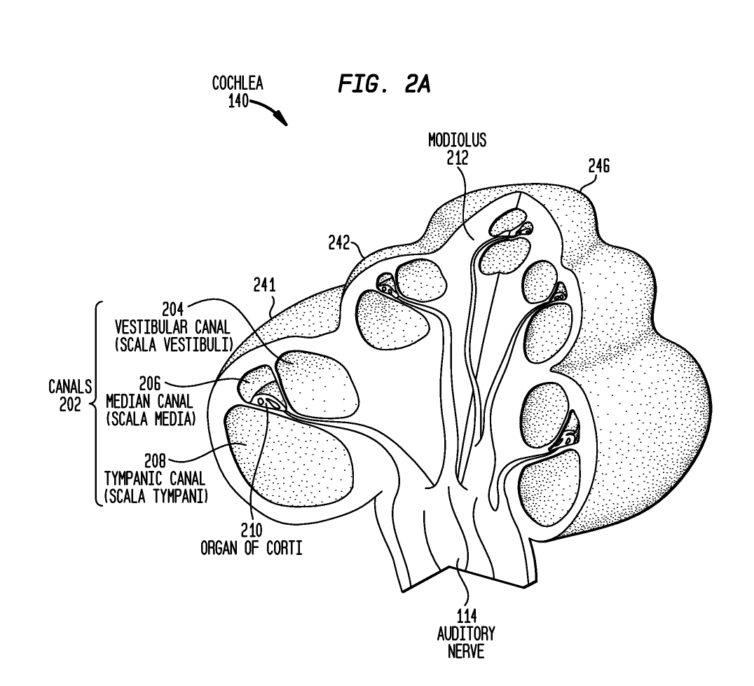

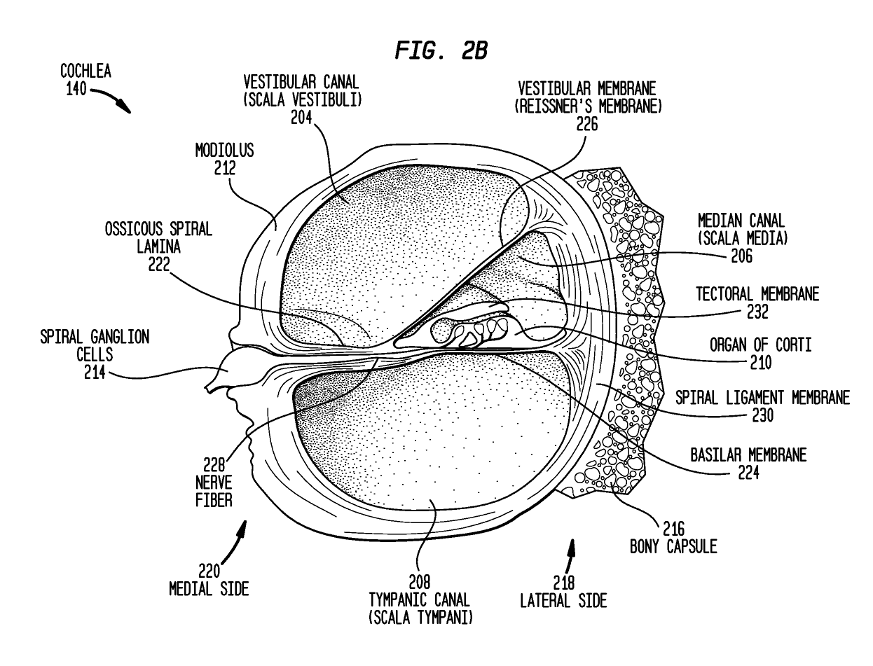

Relevant aspects of cochlea 140 are described next below with reference to FIGS. 2A-2C. FIG. 2A is a perspective view of cochlea 140 partially cut-away to display the canals and nerve fibers of the cochlea. FIG. 2B is a cross-sectional view of one turn of the canals of cochlea 140. To facilitate understanding, the following description will reference the cochlea illustrated in FIGS. 2A and 2B as cochlea 140, which was introduced above with reference to FIG. 1.

Referring to FIG. 2A, cochlea 140 is a conical spiral structure comprising three parallel fluid-filled canals or ducts, collectively and generally referred to herein as canals 202. Canals 202 comprise the tympanic canal 208, also referred to as the scala tympani 208, the vestibular canal 204, also referred to as the scala vestibuli 204, and the median canal 206, also referred to as the scala media 206. Cochlea 140 has a conical shaped central axis, the modiolus 212, that forms the inner wall of scala vestibuli 204 and scala tympani 208. Tympanic and vestibular canals 208, 204 transmit pressure, while medial canal 206 contains the organ of corti 210 which detects pressure impulses and responds with electrical impulses which travel along auditory nerve 114 to the brain (not shown).

Cochlea 140 spirals about modiolus 212 several times and terminates at cochlea apex 134. Modiolus 212 is largest near its base where it corresponds to first turn 241 of cochlea 140. The size of modiolus 212 decreases in the regions corresponding to medial 242 and apical turns 246 of cochlea 140.

Referring now to FIG. 2B, separating canals 202 of cochlear 140 are various membranes and other tissue. The Ossicous spiral lamina 222 projects from modiolus 212 to separate scala vestibuli 204 from scala tympani 208. Toward lateral side 218 of scala tympani 208, a basilar membrane 224 separates scala tympani 208 from scala media 206. Similarly, toward lateral side 218 of scala vestibuli 204, a vestibular membrane 226, also referred to as the Reissner's membrane 226, separates scala vestibuli 204 from scala media 206.

Portions of cochlea 140 are encased in a bony capsule 216. Bony capsule 216 resides on lateral side 218 (the right side as illustrated in FIG. 2B), of cochlea 140. Spiral ganglion cells 214 reside on the opposing medial side 220 (the left side as illustrated in FIG. 2B) of cochlea 140. A spiral ligament membrane 230 is located between lateral side 218 of spiral tympani 208 and bony capsule 216, and between lateral side 218 of scala media 206 and bony capsule 216. Spiral ligament 230 also typically extends around at least a portion of lateral side 218 of scala vestibuli 204.

The fluid in tympanic and vestibular canals 208, 204, referred to as perilymph, has different properties than that of the fluid which fills scala media 206 and which surrounds organ of Corti 210, referred to as endolymph. Sound entering auricle 110 causes pressure changes in cochlea 140 to travel through the fluid-filled tympanic and vestibular canals 208, 204. As noted, organ of Corti 210 is situated on basilar membrane 224 in scala media 206. It contains rows of 16,000-20,000 hair cells (not shown) which protrude from its surface. Above them is the tectoral membrane 232 which moves in response to pressure variations in the fluid-filled tympanic and vestibular canals 208, 204. Small relative movements of the layers of membrane 232 are sufficient to cause the hair cells in the endolymph to move thereby causing the creation of a voltage pulse or action potential which travels along the associated nerve fiber 228. Nerve fibers 228, embedded within spiral lamina 222, connect the hair cells with the spiral ganglion cells 214 which form auditory nerve 114. As explained below, the action potential is relayed via neural pathways through the auditory nerve 114 to the auditory areas of the brain (not shown) for processing.

The place along basilar membrane 224 where maximum excitation of the hair cells occurs determines the perception of pitch and loudness according to the place theory. Due to this anatomical arrangement, cochlea 140 has characteristically been referred to as being “tonotopically mapped.” That is, regions of cochlea 140 toward basal region 116 are responsive to high frequency signals, while regions of cochlea 140 toward apical end 116 are responsive to low frequency signals. These tonotopical properties of cochlea 140 are exploited in a cochlear implant by delivering stimulation signals within a predetermined frequency range to a region of the cochlea that is most sensitive to that particular frequency range.

As is well known in the art, the human auditory system is composed of many structural components, some of which are connected extensively by neural pathways comprising bundles of nerve cells (neurons). Each nerve cell has a cell membrane which acts as a barrier to prevent intercellular fluid from mixing with extracellular fluid. The intercellular and extracellular fluids have different concentrations of ions, which leads to a difference in charge between the fluids. This difference in charge across the cell membrane is referred to herein as the membrane potential (Vm) of the nerve cell. Nerve cells use membrane potentials to transmit signals between different parts of the auditory system.

In nerve cells that are at rest (i.e., not transmitting a nerve signal) the membrane potential is referred to as the resting potential of the nerve cell. Upon receipt of a stimulus, the electrical properties of a nerve cell membrane are subjected to abrupt changes, sometimes referred to herein as a nerve impulse or nerve action potential. The action potential represents the transient depolarization and repolarization of the nerve cell membrane. The action potential causes electrical signal transmission along the conductive core (axon) of a nerve cell. Signals may be then transmitted along a group or population of nerve cells via such propagating action potentials.

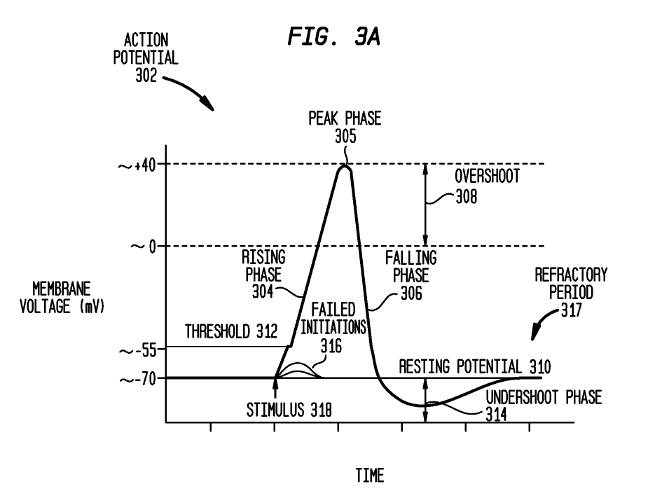

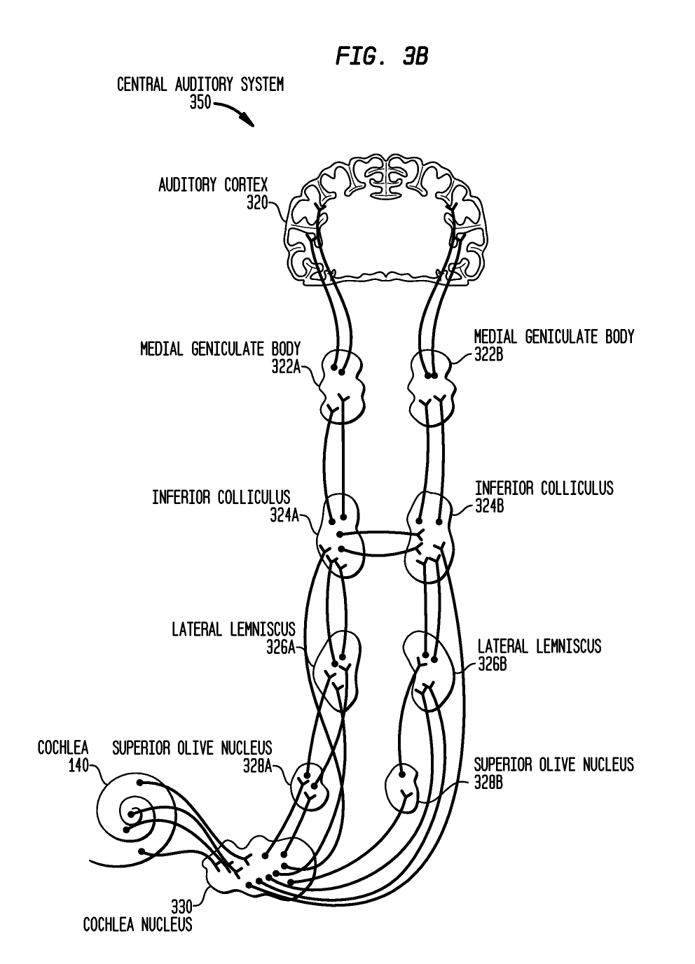

FIG. 3A is graph illustrating the various phases of an idealized action potential 302 as the potential passes through a nerve cell in accordance with embodiments of the present invention. The action potential is presented as membrane voltage in millivolts (mV) versus time. As would be appreciated by one of ordinary skill in the art, the membrane voltages and times shown in FIG. 3 are provided for illustration purposes only. The actual voltages may vary depending on the individual. As such, this illustrative example should not be construed as limiting the present invention.

In the example of FIG. 3, prior to application of a stimulus 318 to the nerve cell, the resting potential of the nerve cell is approximately −70 mV. Stimulus 318 is applied at a first time. In normal hearing, this stimulus is provided as a result of the movement of the hair cells of the cochlea. Movement of these hair cells results in the generate of a nerve impulse.

As shown in FIG. 3, following application of stimulus 318, the nerve cell begins to depolarize. Depolarization of the nerve cell refers to the fact that the voltage of the cell becomes more positive following stimulus 318. When the membrane of the nerve cell becomes depolarized beyond the cell's critical threshold, the nerve cell undergoes an action potential. This action potential is sometimes referred to as the “firing” of the nerve cell. As used herein, the critical threshold of a nerve cell, group of nerve cells, etc. refers to the threshold level at which the nerve cell, group of nerve cells, etc. will undergo an action potential. In the example illustrated in FIG. 3, the critical threshold level for firing of the nerve cell is approximately −50 mV. As would be appreciated, the critical threshold and other transitions may be different for various recipients. As such, the values provided in FIG. 3 are merely illustrative. For consistency, a critical threshold of −50 mV will be used herein, but such usage should not be considered to limit the present invention

The course of this action potential in the nerve cell can be generally divided into five phases. These five phases are shown in FIG. 3 as a rising phase 304, a peak phase 305, a falling phase 306, an undershoot phase 314, and finally a refractory period 317. During rising phase 304, the membrane voltage continues to depolarize. The point at which depolarization ceases is shown as peak phase 305. In the illustrative embodiment of FIG. 3, at this peak phase 305, the membrane voltage reaches a maximum value of approximately 40 mV.

Following peak phase 305, the action potential undergoes falling phase 306. During falling phase 306, the membrane voltage becomes increasingly more negative, sometimes referred to as hyperpolarization of the nerve cell. This hyperpolarization causes the membrane voltage to temporarily become more negatively charged then when the nerve cell is at rest. This phase is referred to as the undershoot phase 314 of action potential 302. Following this undershoot, there is a time period during which it is impossible or difficult for the nerve cells to fire. This time period is referred to as refractory period 317.

Action potential 302 illustrated in FIG. 3 may travel through, for example the auditory system, without diminishing or fading out because the action potential is regenerated at each nerve cell. This regeneration occurs because an action potential at one nerve cell raises the voltage at adjacent nerve cells. This induced rise in voltage depolarizes adjacent nerve cells thereby provoking a new action potential therein.

As noted above, the nerve cell must obtain a membrane voltage above a critical threshold before the nerve cell may fire. Illustrated in FIG. 3 are several failed initiations 316 which occur as a result of stimuli which were insufficient to raise the membrane voltage above the critical threshold value to result in an action potential.

A cellular structure known as a synapse is used to transfer an action potential from a first nerve cell to an adjacent nerve cell. A synapse is the junction between two neurons. When an action potential within a first nerve cell reaches a synapse, neurotransmitters are released which bind with neurotransmitter receptacles in the adjacent nerve cell. The received neurotransmitter commences an action potential in the receiving nerve cell.

As is well known, thousands of nerve cells are used to sense and relay information in the human auditory system. Although the above discussion has discussed the relay of a signal using the simplified example of a single action potential, it would be appreciated that received information would be collected and relayed to the brain for processing using a large number of nerve cells. Thus, a network of interacting nerve cells is required for a full spectrum of information to be collected and transferred to the brain.

The human auditory system may be divided into two large subsystems, namely the peripheral auditory system and the central auditory system. The peripheral auditory system comprises outer ear 101 (FIG. 1), the middle ear 105 (FIG. 1), and the inner ear 107 (FIG. 1). As explained above with reference to FIG. 1, a sound 103 is collected by outer ear 101 and channeled into and through ear canal 102. Disposed across the distal end of ear cannel 102 is a tympanic membrane 104 which vibrates in response to sound wave 103. This vibration is coupled to oval window or fenestra ovalis 112 through ossicles 106. Ossicles 106 causes oval window 112 to articulate, or vibrate in response to vibration of tympanic membrane 104. This vibration sets up waves of fluid motion of the perilymph within cochlea 140. Such fluid motion, in turn, activates tiny hair cells (not shown) inside cochlea 140. Activation of the hair cells causes appropriate action potentials (nerve impulses) to be generated. These nerve impulses are transferred via neural pathways through the recipient's central auditory system to the brain where they are perceived as sound. FIG. 3B is a schematic diagram of a recipient's central auditory system 350 through which sensed auditory information may be relayed to the brain.

It should be appreciated that FIG. 3B is a highly schematic and simplified diagram that illustrates only the main tracts and nuclei of central auditory system 350, although other nuclei exist. Therefore, the components of central auditory system 350, which include superior olive nuclei 328, lateral lemniscus 326, inferior colliculi 324, medial geniculate body 322 and auditory cortex 320, are shown schematically and do not provide represent the physical organization of the auditory system.

Central auditory system 350 forms a bilateral auditory pathway where signals from both cochlea are delivered to different sides of the brain. This is represented by the two parallel paths of FIG. 3B. It is well known that once an impulse leaves cochlear nucleus 330, the contralateral (opposite) path relays signals to the brain. In other words, most of the auditory information processed by each half of the brain comes from the ear on the other side of the head.

As noted, nerve impulses are generated at cochlea 140 and are relayed first to cochlear nucleus 330. The impulses are relayed from cochlear nucleus 330 to the superior olivary complex of the brainstem which comprises superior olive nuclei 328. From superior olive nuclei 328, the impulses are relayed through and/or around brainstem lateral lemniscus 326 to inferior colliculi 324 of the midbrain. The impulses are further relayed through the medial geniculate body 322 of the thalamic relay system to auditory cortex 320 where the impulses are perceived as sound.

The ability of a cochlear implant to restore a recipient's hearing depends largely one the proper functioning of central auditory system 350, and the ability of system 350 to relay impulses to auditory cortex 320. Central auditory system 350 is a dynamic system in which there is a constant change in the way the nerve cells are connected or arranged to allow transfer or relay of impulses. In other words, the central auditory system 350 changes or adapts as a result of new conditions or experiences. The ability of central auditory system 350, and a recipient's neural system in general, to adapt to new conditions or experiences is referred to herein as neural plasticity.

The neural plasticity of central auditory system 350 may be problematic in a cochlear implant recipient because the cochlear implant causes dramatic changes in how a sound is received when compared to a person with undamaged hearing. For example, cochlear implant recipients typically receive impulses from one ear only, rather than from two ears. Also, the cochlear implant has a finite number of locations at which stimulation signals are delivered as compared to a fully functional ear in which received auditory signals are processed by between 3000-4000 hair cells. These changes may cause neural rearrangements that negatively affect the ability of central auditory system 350 to effectively relay impulses to auditory cortex 320.

Embodiments of the present invention are directed to controlling, manipulating or modulating the neural plasticity of the recipient's central auditory system 350. Embodiments of the present invention modulate the neural plasticity of auditory system 350 by applying pharmaceutical agents to the recipient's cochlea nerve cells which influence and/or control the neural plasticity of the system. The applied pharmaceutical agents which influence and/or control the neural plasticity system are referred to herein as plasticity modulating agents. As described in greater detail below, the plasticity modulating agents are configured to induce an action potential which is relayed to the brain, but which does not give rise to hearing percept by the recipient.

As noted, to evoke a hearing percept, a cochlear implant applies electrical stimulation signals to the recipient's cochlea nerve cells. The electrical stimulation signals generate impulses (action potentials) in the stimulated nerve cells which are relayed to the brain where the impulses result in the sensation of sound. The impulses are most readily received by the brain via active neural pathways. In embodiments of the present invention, the plasticity modulating agents are applied to induce certain desired or selected neural pathways within the central auditory system to remain active. The plasticity modulating agents are used to reinforce pathways that are going to relay impulses configured to evoke a hearing sensation by the brain. This may enhance the effectiveness of applied electrical stimulation signals representing a sound signal, and thereby lead to improved speech coding strategies

In certain embodiments, the ability to modulate the neural plasticity may provide a clinician or other user with the ability to tune the response of the central auditory system to electrical stimulation signals representing a sound signal. Specifically, embodiments of the present invention may provide a clinician with the ability to use plasticity modulating agents to train the central auditory system to respond in a desired or predicted manner to the application of stimulation signals representing a sound signal.

In additional embodiments of the present invention, a plasticity modulating agent is applied to the recipient's cochlea in order to provide a substantially uniform response of the cochlea to electrical stimulation signals. Specifically, a plasticity modulating agent is applied to cochlear nerve cells at one or more locations along the cochlea. The plasticity modulating agent adjusts or modifies the response of those nerve cells to electrical stimulation to match the response of other nerve cells located at a different region of the cochlea. Therefore, the same electrical stimulation applied to different cochlea regions will have the same effect on the stimulated nerve cells. This ability to standardize the cochlea's response to electrical stimulation signals may provide for enhanced stimulation strategies.

As used herein, a plasticity modulating pharmaceutical agent refers to any artificial or naturally occurring drug, medicine, pharmaceutical, hormone suitable to influence and/or control the neural plasticity of the recipient's auditory system. In embodiments of the present invention, the plasticity modulating agent may comprise an agent that causes a desired response (referred to as a desired agent), or a precursor for a desired agent. The precursor for the desired agent can be in a form similar to that of the desired agent which undergoes a chemical, physical or biological change to take the form of the desired agent, or may cause formation of the desired agent. For example, an applied gene is not the desired agent, but activation of the gene produces a desired agent.

In certain embodiments, the plasticity modulating agent comprises a naturally occurring agent such as a neurotrophic factor or a neurotrophin. In certain embodiments, the neurotrophic factors may comprise Brain Derived Neurotrophic Factor (BDNF). In other embodiments, the neurotrophic factors may be selected from the group comprising, but not limited to, NGF, NT-3, NT-4/5, NT-6, LIF, GDNF, CNTF, and IGF-I. In further embodiments of the present invent, the naturally occurring agents can comprise one of more factors, other then neurotrophins, which have a capacity to activate neurotrophic receptors of the nerve cells, such as, for example adenosine or a neuromodulator.

Neurotrophic factors are a key element in establishment and maintenance of synapses. Specifically, in the absence of signals, synaptic contacts between nerve cells may disconnect, breaking a particular neural pathway. Details of the cellular functions relating to neurotropic factors may be found in commonly-owned and co-pending U.S. patent application Ser. No. 10/494,995, from which this application claims priority. The content of this application is hereby incorporated by reference herein.

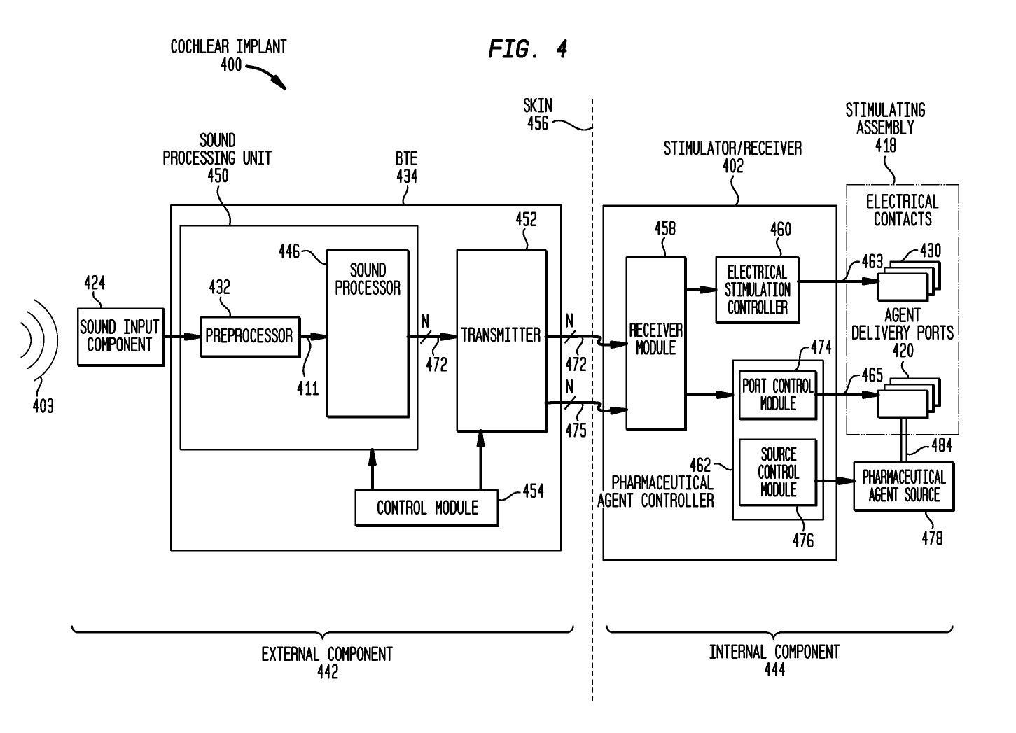

Plasticity modulating agents in accordance with embodiments of the present invention are applied to a recipient's cochlea nerve cells. FIG. 4 is a detailed functional block diagram of a cochlear implant 400 that may be used to implement the above and other embodiments of the present invention. As shown, elements of cochlear implant 400 that have substantially the same or similar structures and/or perform substantially the same or similar functions as elements of cochlear implant 100 are illustrated in FIG. 4 using a 400 series reference number having two right digits which are the same as the right two digits as the corresponding element of FIG. 1. For example, as shown, cochlear implant 400 comprises an embodiment of external component 142 of FIG. 1, referred to as external component 442.

In the illustrative embodiment of FIG. 4, external component 442 comprises a behind-the-ear (BTE) device 434 and one or more sound input components 424. Sound input component 424 is configured to receive a sound signal 203. Sound input component 424 may comprise, for example, one or more microphones, a telecoil, or an electrical input which connects cochlear implant 400 to FM hearing systems, MP3 players, musical instruments, computers, televisions, mobile phones, etc. As such, sound signal 403 may comprise a sound wave or an electrical audio signal. In the embodiment of FIG. 4, sound input component 424 comprises a microphone 424 which may be a directional microphone and/or an omni-directional microphone. Sound input component 424 outputs signals 409 representing received sound signal 403 to sound processing unit 450 within BTE 434.

BTE 434 is configured to be worn behind the ear of the recipient and, as described herein, may comprise various sound processing and other components. Microphone 424 may be positionable on BTE 434 or elsewhere on the recipient.

As would be appreciated by those of ordinary skill in the art, although the embodiments of FIG. 4 are described with reference to external component 442 configured as a BTE, other configurations of external component 442 may also be implemented in embodiments of the present invention. For example, in certain embodiments, external component 442 may be configured as a body-worn sound processing unit instead of, or in combination with, a component that is worn behind the ear. In other embodiments, external component 442 may be omitted and microphone 424 as well as the components residing in BTE device 434 may be implanted in the recipient. Such an arrangement of a cochlear implant is sometimes referred to as a totally-implantable cochlear implant. For ease of description, embodiments of the present invention will be primarily described herein with reference to cochlear implants having external components. However, embodiments of the present invention may be equally implemented in any cochlear implant now known or later developed.

BTE device 434 comprises a sound processing unit 450, a transmitter 452 and a control module 454. As noted above, microphone 424 receives a sound signal and delivers corresponding electrical signals 409 to a preprocessor 432 of sound processing unit 450. Prep-processor 432 may comprise various combinations of preamplifiers, automatic gain controllers, and Analog-to Digital-Converters used to convert signal 409 in a digital signal 411 for use by sound processor 446.

As would be appreciated, in certain embodiments of the present invention, pre-processor 432 may be implemented as a component of sound input component 424. It should also be appreciated that in certain embodiments, one or more components of pre-processor module 432 may not be necessary. For example, in certain embodiments, sound signal 403 received by sound input component 424 comprises a digitized signal received from, for example, a FM hearing system, MP3 player, television, mobile phones, etc. In these embodiments, the received signal may be provided directly to sound processor 446.

Sound processor 446 performs sound processing operations to convert electrical signals 411 received from preprocessor 432 into one or more encoded data signals 472 which are then transmitted to internal component 444 by transmitter 452. There are numerous strategies that may be implemented by sound processor 446 to convert signals 411 into encoded data signals 472. Embodiments of the present invention may be used in combination with any processing strategy now or later developed.

Embodiments of cochlear implant 400 may locally store several processing strategies as a software program or otherwise, any one of which may be selected depending, for example, on the recipient's listening environment. For example, a recipient may choose one strategy for a low noise environment, such as a conversation in an enclosed room, and a second strategy for a high noise environment, such as on a public street. The programmed speech strategies may be different versions of the same speech strategy, each programmed with different parameters or settings.

External component 442 may further comprise a control module 454. Control module 454 may be configured to receive control inputs from a recipient, an external device, or internally generated events, commands or interrupts. Control module 454 controls sound processing unit 450 and/or transmission of signals to internal component 444. As described below, in one embodiment, control module causes a control signal 475 to be transmitted to internal component 444.

In the embodiments illustrated in FIG. 4, internal component 444 comprises a stimulator/receiver unit 402, a stimulating assembly 418 and a pharmaceutical agent source 478. Stimulator/receiver unit 402 comprises a receiver module 458 that receives from transmitter 452 encoded data signals 472 and control signal 475. Stimulator/receiver unit 402 includes an electrical stimulation controller 460 that generates electrical stimulation signals 463 which are applied to the recipient via electrical contacts 430 of stimulating assembly 418. Electrical stimulation controller 460 generates electrical stimulation signals 463 based on encoded data signals 472 and cause perception of sound signal 403 by the recipient.

As noted, internal component comprises agent pharmaceutical agent source 478 which is fluidically coupled to agent delivery ports 420 in stimulating assembly 418. Pharmaceutical agent source 478 is configured to store a plasticity modulating agent therein. A plasticity modulating agent released by pharmaceutical agent source 478 is applied to the nerve cells of the recipient's cochlea via agent delivery ports 420. Stimulator/receiver unit 402 includes a pharmaceutical agent controller 462 that controls the delivery of the plasticity modulating agent to the recipient's cochlea nerve cells. Specifically, pharmaceutical agent controller 462 comprises a port control module 474 to control ports 420 via, for example, electrical signals 465, and a source control module 476 to control the release of the plasticity modulating agent from pharmaceutical agent source 478 to stimulating assembly 418. In the illustrative embodiment of FIG. 4, a coupling member, such as a catheter or tube 484, fluidically couples pharmaceutical agent source 478 to ports 420 in stimulating assembly 418.

As described below, in certain embodiments, pharmaceutical agent controller 462 causes a plasticity modulating agent to be applied based on control signal 475. In certain embodiments, stimulator/receiver unit 402 may cause concurrent application of the plasticity modulating agent and electrical stimulation signals 463.

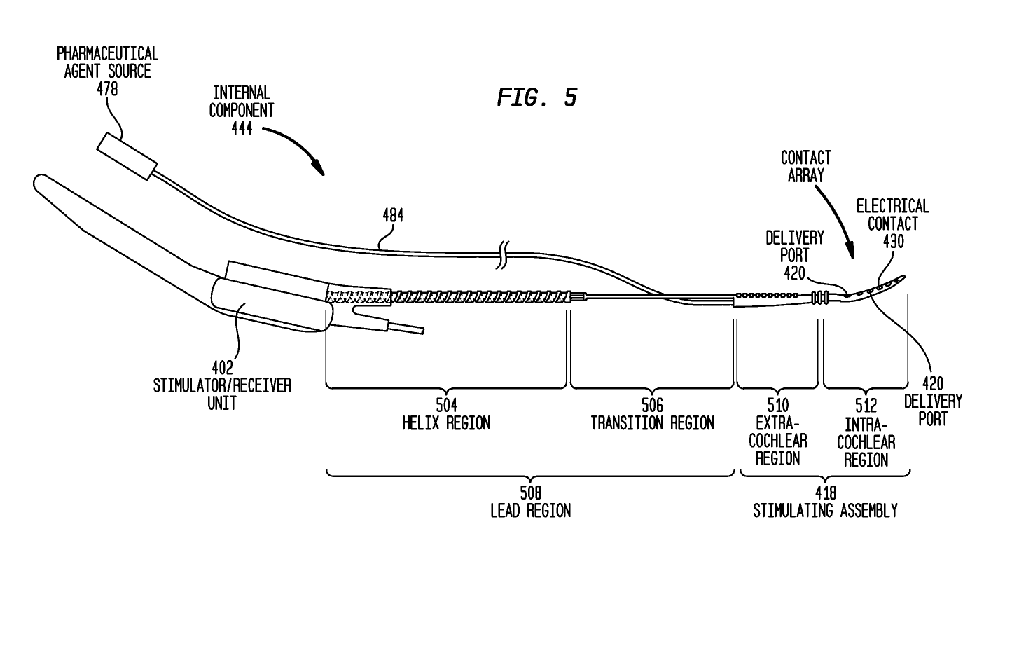

FIG. 5 is a simplified side view of an embodiment of internal component 444. As noted, internal component 444 comprises a stimulator/receiver unit 402 which, as described above, receives encoded signals from an external component of the cochlear implant. Internal component 444 terminates in a stimulating assembly 418 that comprises an extra-cochlear region 510 and an intra-cochlear region 512. Intra-cochlear region 512 is configured to be implanted in the recipient's cochlea and has disposed thereon an array 516 of electrical contacts. Stimulating assembly 418 further includes one or more delivery ports 420.

In certain embodiments, stimulating assembly 418 is configured to adopt a curved configuration during and or after implantation into the recipient's cochlea. To achieve this, in certain embodiments, stimulating assembly 418 is pre-curved to the same general curvature of a recipient's cochlea. In such embodiments, of stimulating assembly 418 is sometimes referred to as perimodiolar stimulating assembly and is typically held straight by, for example, a stiffening stylet (not shown) which is removed during implantation so that the stimulating assembly may adopt its curved configuration when in the cochlea. Other methods of implantation, as well as other stimulating assemblies which adopt a curved configuration, may be used in alternative embodiments of the present invention.

In other embodiments, stimulating assembly 418 is a non-perimodiolar stimulating assembly which does not adopt a curved configuration. For example, stimulating assembly 418 may comprise a straight stimulating assembly or a mid-scala assembly which assumes a mid-scala position during or following implantation. In further embodiments, cochlear implant 400 could include a stimulating assembly implantable into a natural crevice in the cochlea that allows for the hydrodynamic nature of the cochlea to be maintained, or an assembly positioned adjacent to the cochlea.

As noted above, embodiments of the present invention sequentially or concurrently apply a plasticity modulating agent and electrical stimulating signals that evoke a hearing percept. In embodiments of the present invention, cochlear implant 400 comprises two stimulating assemblies. A first stimulating assembly is used to apply a plasticity modulating agent, while the second stimulating assembly is used to apply electrical stimulating signals that evoke a hearing percept. One or both of these stimulating assemblies may be positioned outside of the recipient's cochlea.

Internal component 444 further comprises a lead region 508 coupling stimulator/receiver unit 402 to stimulating assembly 418. Lead region 508 comprises a helix region 504 and a transition region 506. Helix region 504 is a section of lead region 508 in which electrical leads are would helically. Transition region 506 connects helix region 504 to stimulating assembly 418. Electrical stimulation signals generated by stimulator/receiver unit 402 are applied to contact array 416 via lead region 508. Helix region 504 prevents lead region 508, and thus the connection between stimulator/receiver 402 and stimulating assembly 418, from being damaged due to movement of internal component 444 which may occur, for example, during mastication.

As detailed above with reference to FIG. 4, stimulator/receiver unit 402 includes an electrical stimulation controller 460 (not shown) and a pharmaceutical agent controller 462 (also not shown). Pharmaceutical agent controller 462 controls the delivery of a plasticity modulating agent from pharmaceutical agent source 478 to the cochlea. Specifically, agent controller 462 cause delivery of a plasticity modulating agent to the cochlea in various temporal and spatial patterns and profiles, for example, by releasing the plasticity modulating agent in a continuous or pulsatile manner, and/or targeting areas of the cochlea. As described below, this delivery control is provided by controlling one or more of pharmaceutical agent source 478 and delivery ports 420.

In the embodiments of FIG. 5, pharmaceutical agent source 478 is physically separate from stimulator/receiver unit 402. Pharmaceutical agent source 478 is coupled to stimulating assembly 418 via a catheter or tube 484. For example, in embodiments of the present invention, pharmaceutical agent source 478 comprises a reservoir (not shown) for storing a plasticity modulating agent. Pharmaceutical agent source 478 may be located underneath and proximate or adjacent to the recipient's skin so that the reservoir may be refilled when the agent therein is depleted. For example, the reservoir may include a post-operatively accessible refill port configured to receive a syringe therein. The syringe provides the plasticity modulating agent via an injection through the skin.

In certain embodiments of the present invention, pharmaceutical agent source 478 comprises an active infusion device. Such an active infusion device includes a plasticity modulating agent reservoir, a peristaltic pump to pump the agent from the reservoir, and a catheter port to connect pharmaceutical agent source 478 to a catheter. The catheter extends from agent source 478 to stimulating assembly 418. Pharmaceutical agent source 478 in accordance with such embodiments of the present invention may also include a battery to power the pump, an electronic module to control the flow rate of the pump, and possibly an antenna to permit the remote programming or control of the pump. It should be appreciated that agent source 478 may be secured internally or externally to the recipient.

In alternative embodiments of the present invention, pharmaceutical agent source 478 comprises a passive infusion device that does not include a pump. In such embodiments, pharmaceutical agent source 478 includes a pressurized reservoir that delivers the plasticity modulating agent to stimulating assembly 418 via a catheter. The pressurization of the reservoir is provided by a syringe capable of delivering a plasticity modulating agent to the reservoir.

As detailed below, in alternative embodiments of the present invention, pharmaceutical agent source 478 may be integrated in stimulator/receiver unit 402 or stimulating assembly 418. In such embodiments, pharmaceutical agent source 478 comprises a reservoir within stimulating assembly 418. The reservoir may be connected to a post-operatively accessible refill element. The post-operatively accessible refill element may comprise an additional reservoir positioned underneath the skin as described above, or the refill element may comprise a refill port positioned underneath and proximate to the skin. In both cases, the refill element may be connected to the reservoir in stimulating assembly 418 via a catheter.

In embodiments of the present invention, delivery ports 420 are controllable to alter the flow rate through the ports. Such control may be provided by the implant, or externally, via, for example, electrical or mechanical signals, heat, etc.

Other systems and methods for delivering a plasticity modulating agent are within the scope of the present invention. For example, in one embodiment, plasticity modulating agent is stored as a hydrogel rather than as a fluid within a reservoir.

As noted above, embodiments of the present invention are generally directed to a cochlear implant configured to apply combinations of electrical stimulation signals and plasticity modulating agents to a recipient's cochlea nerve cells. As discussed in greater detail below, the cochlear implant is configured to control the timing, location, etc. of the delivery in order to cause a desired effect on central auditory system 350. FIG. 6 is a cross-sectional side view of a portion of an elongate stimulating assembly 618 which may be used in accordance with embodiments of the present invention to apply plasticity modulatining agents and electrical stimulation signals to the recipient's cochlea. As noted above, elongate stimulating assembly 618 has a proximal end connected to a stimulator/receiver unit (not shown) and a distal end implantable in a recipient's cochlea. FIG. 6 illustrates a portion that is implantable into the cochlea.

Stimulating assembly 618 comprises a longitudinally aligned and distally extending array 646 of stimulating electrical contacts 630. Electrical contacts 630 receive electrical stimulation signals from the receiver/stimulator unit via one or more wires (not shown). The received electrical stimulation signals are then applied to the recipient's cochlea nerve cells. Stimulating assembly 618 further includes a plurality of pharmaceutical agent delivery ports 620 to apply plasticity modulating agents to the cochlea nerve cells. In this exemplary arrangement, delivery ports 620 include channels 622 which fluidically couple the ports to a pharmaceutical agent source, shown in FIG. 6 as agent reservoir 678 within stimulating assembly 618.

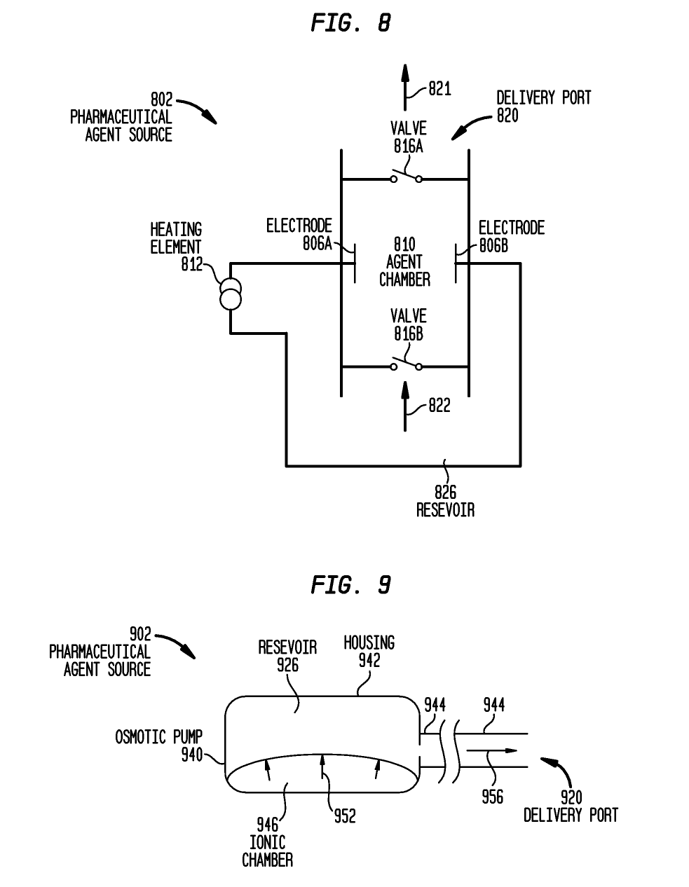

In certain embodiments, reservoir 678 may be coupled to an additional reservoir or refill element positioned outside of the cochlea. As noted, the stimulator/receiver unit includes one or more modules to control the operation of delivery ports 620, channel 622 and/or a pump (not shown) connected to reservoir 678 in order to control the application of the plasticity modulating agent to the recipient. For example, in one embodiment, the flow rate through delivery ports is electrically controllable. In other embodiments, channels 622 comprise controllable ion channels. FIGS. 8 and 9 illustrate exemplary arrangements for controlling a pharmaceutical agent source.

In embodiments of the present invention, the amount of plasticity modulating agent applied, as well as the concentration of the agent, may impact the response of the nerve cells to the agent. Thus, the amount of agent released from different delivery ports 620 may be different depending on the desired application and the properties of the nerve cell population proximate to a delivery port. Therefore, the application of the plasticity modulating agent may be uniform along the cochlea, or place and/or time specific. As described elsewhere herein, cochlear implant 400 is configured to control the location and timing of the plasticity modulating agent.

Embodiments of the present invention may also deliver two or more different plasticity modulating agents to the cochlea. The different drugs may be delivered sequentially or concurrently, and may be delivered to the same or different regions of the cochlea.

Furthermore, in alternative embodiments of the present invention, the concentration of the plasticity modulating agent, or the agent itself, can differ between delivery ports 620. In such embodiments, the pharmaceutical agent source may be configured to deliver different agents or different concentrations of agents to different ports.

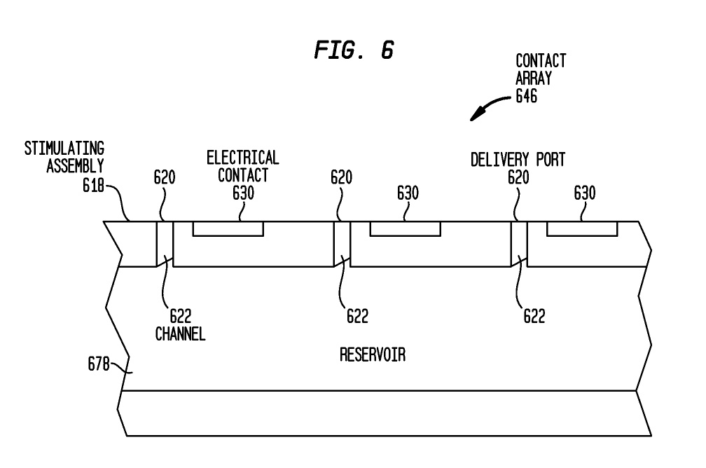

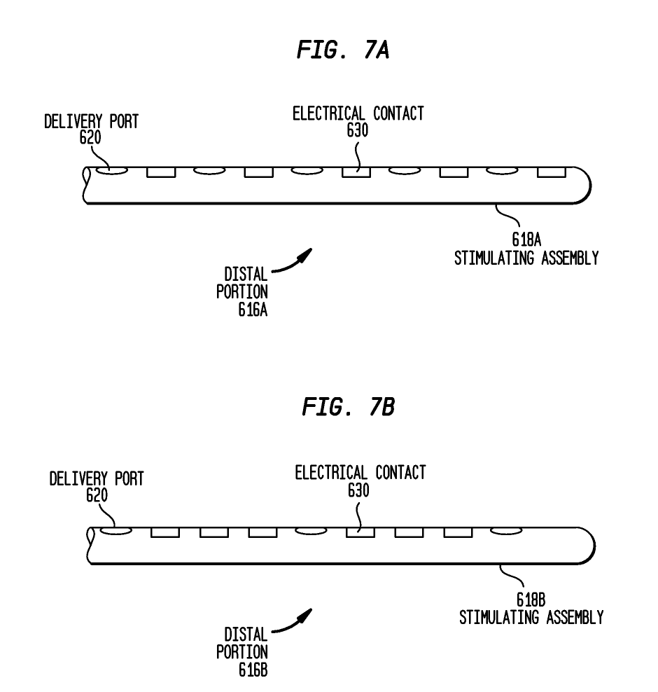

FIG. 6 illustrates embodiments of the present invention in which delivery ports 620 are each disposed adjacent an electrical contact 630. It will be understood that the number and placement of the delivery ports 620 and electrical contacts 630 can be varied without departing from the scope of the present invention. FIGS. 7A and 7B are side views of stimulating assembly 618 illustrating alternative arrangements of delivery ports 620 and electrical contacts 630. For ease of illustration, electrical contacts 630 are depicted as rectangles and delivery ports 620 are depicted as ovals. These exemplary shapes are provided only to facilitate understanding of embodiments of the present invention and do not define or limit electrical contacts 630 or delivery ports 620 in any manner.

In FIG. 7A, a distal portion 616A of a stimulating assembly 618A is illustrated. As shown, electrical contacts 630 and delivery ports 620 are arranged in an alternating fashion. In other words, in the illustrated arrangement of FIG. 7A, no delivery ports 620 are adjacent other delivery ports. Similarly, no electrical contacts 630 are adjacent other electrical contacts. In contrast, as shown in FIG. 7B, a smaller number of delivery ports 620 are dispersed along distal portion 616B.

FIG. 8 is a diagram illustrating a pharmaceutical agent source 802 for delivering pulses of a plasticity modulating agent to a recipient's cochlea in accordance with embodiments of the present invention. As shown, pharmaceutical agent source 802 comprises a reservoir 826 coupled to an outlet port, such as a delivery port 820. Disposed between reservoir 826 and delivery port 820 is an agent chamber 810 bound by two unidirectional valves 816. Agent chamber 810 is substantially filled with a plasticity modulating agent. In operation, pulses or cycles of electrical current are applied to opposing electrodes 806 in agent chamber 810 via heating element 812. Application of the electrical current to electrodes 806 that results in rapid heating and expansion of the agent within agent chamber 810 through, for example, thermal expansion and/or cavitation as the pharmaceutical agent near electrodes 806 boil. This expansion forces an amount of the agent in chamber 810 through unidirectional valve 816A towards delivery port 820. This is shown by arrow 821. Unidirectional valve 816B prevents the flow of the plasticity modulating agent towards reservoir 822. After the delivery of electrical current to electrodes 806 is stopped, the pharmaceutical agent within chamber 810 cools and contracts. This contraction draws additional agent from reservoir 826 through unidirectional valve 416B into chamber 810, shown by arrow 822.

As would be appreciated, the embodiments illustrated in FIG. 8 are used for plasticity modulating agents that do not experience property changes as the result of the application of heat thereto. If a heat sensitive plasticity modulating agent is used, the above described valve and chamber system may still be used, but heating element 812 is replaced with an electro-mechanical actuator which, when activated, is configured to change the physical volume of chamber 810. In such embodiments, the walls of agent chamber 810 are flexible so that the actuator contracts chamber 810 to force plasticity modulating agent out of unidirectional valve 816A, and expands chamber 810 to draw agent in from reservoir 826. Suitable electro-mechanical actuators include piezoelectric devices, coil and magnet systems and electret devices. In both of the systems described with reference to FIG. 8, the amount of plasticity modulating agent applied to the cochlea can be controlled by controlling the amount and rate of the driving current applied to the chamber or actuator.

In the illustrative embodiment of FIG. 8, pharmaceutical agent source 802 is shown in close proximity to delivery port 820. It would be appreciated that source 802 is not necessarily close to delivery port 820. For example, a pharmaceutical agent released from valve 816A may be provided to a catheter connecting system 802 to delivery port 820.

Furthermore, in certain embodiments, a single pharmaceutical agent source 802 may be provided to deliver a plasticity modulating agent to multiple delivery ports 820. In alternative embodiments, multiple pharmaceutical agent sources 802 may be provided.

FIG. 9 is a diagram illustrating a pharmaceutical agent source 902 for delivering pulses of a plasticity modulating agent to a recipient's cochlea in accordance with embodiments of the present invention. As shown, pharmaceutical agent source 902 comprises an osmotic pump 940 to provide a plasticity modulating agent to a delivery port 920 via catheter 944.

As shown, osmotic pump 940 includes a housing 942 having a flexible reservoir 926 containing a plasticity modulating agent therein. A second portion of housing 942 comprises an ionic chamber 946 that contains a fluid having a lower ionic concentration that fluid external to housing 942. An osmotic membrane 950 forms a part of housing 942 which separates ionic chamber 946 from the external fluid. Osmotic pump 940 relies upon an osmotic pressure difference between the ionic chamber 946 and the external fluid to release the agent from reservoir 946. Specifically, the osmotic pressure difference causes water to flow into the pump through the semi-permeable osmotic membrane 950. As the water enters ionic chamber 946, ionic chamber 946 exerts a force 952 that compresses flexible reservoir 926, thereby displacing the plasticity modulating agent from reservoir 926.

In the illustrative embodiment of FIG. 9, pharmaceutical agent source 902 is shown in close proximity to delivery port 920. It would be appreciated that source 902 is not necessarily close to delivery port 920. Furthermore, in certain embodiments, a single pharmaceutical agent source 902 may be provided to deliver a plasticity modulating agent to multiple delivery ports 920. In alternative embodiments, multiple pharmaceutical agent sources 902 may be provided.

As noted, plasticity modulating signals may be applied at times and in spatial patterns selected based on various factors. In embodiments of the present invention, plasticity modulating agents applied to the recipient's cochlea nerve cells when cochlear implant 400 is not generating and/or applying electrical stimulation signals configured to evoke a hearing percept. For example, in one such embodiment, cochlear implant 400 could enter a sleep mode in which the cochlear implant does not apply electrical stimulation signals configured to evoke a hearing percept. Cochlear implant 400 could enter such a sleep mode automatically, for example after the implant has not received a sound signal for a predetermined period of time. In other embodiments, cochlear implant 400 could enter a sleep mode based on an input from the recipient. When cochlear implant 400 is in a sleep mode, pharmaceutical agent controller 462 (FIG. 4) may cause application of a plasticity modulating agent for a predetermined period of time and in spatial and temporal patterns determined by the implant or programmed by the recipient, clinician, etc. In embodiments of the present invention, the spatial and temporal patterns for application of a plasticity modulating agent are preprogrammed into the cochlear implant, during, for example, a fitting session.

In other embodiments, cochlear implant 400 is configured to monitor or detect when the implant does not generate and/or apply electrical stimulation signals configured to evoke a hearing percept. In such embodiments, upon determining that no electrical stimulation signals configured to evoke a hearing percept are being applied, or have not been applied for a predetermined period of time, cochlear implant 400 applies a plasticity modulating agent.

In a further embodiment of the present invention, cochlear implant 400 is configured to detect when the transcutaneous transfer of signals from an external component 442 have stopped due to, for example, deactivation of external component 442 or removal of the component from the recipient. When the transcutaneous transfer of signals ceases, pharmaceutical agent controller 462 may cause application of a plasticity modulating agent for application to the cochlea nerve cells.

In embodiments of the present invention, each delivery port may receive and apply various concentrations and amounts of a plasticity modulating agent. The amount and concentration of the agent applied by each delivery port may be the same or different from the concentrations and/or amounts received and applied by other ports. In other words, the delivery of plasticity modulating agents may be place specific. For example, one concentration and/or amount of agent may be applied to the basal turn of the cochlea and another concentration and/or amount may be applied to the apical part of the cochlea.

In embodiments of the present invention, cochlear implant 400 is configured to measure or track the activity of one or more of the electrical contacts during a period of use. During the period of use, cochlear implant 400 measures the frequency that which electrical stimulation signals used to evoke a hearing percept are applied by the electrical contacts. This measurement may be done by measuring the stimulation current, and/or the neural response for each of the one or more electrical contacts. Cochlear implant 400 is then configured to adjust the spatial and/or temporal deliver pattern of a plasticity modulating agent based on the measure of activity during the period of use.

In certain embodiments of the present invention, cochlear implant 400 may operate in an acute or a chronic mode. In the acute mode, the plasticity modulating agent may be delivered to the auditory system over a short period of time when compared to the length of time that cochlear implant 400 is actively evoking hearing percepts. In the chronic mode, the plasticity modulating agent may be presented over the same or comparable period of time as the length of time that the cochlear implant is actively evoking hearing percepts.

As noted above, embodiments of the present invention modulate the neural plasticity of a recipient auditory system by applying plasticity modulating agents to the recipient's cochlea nerve cells which influence and/or control the neural plasticity of the auditory system.

As noted, to evoke a hearing percept, a cochlear implant applies electrical stimulation signals to the recipient's cochlea nerve cells. The electrical stimulation signals generate impulses (action potentials) in the stimulated nerve cells which are relayed to the brain where they impulses result in the sensation of sound. The impulses are most readily received by the brain via active neural pathways. In embodiments of the present invention, the plasticity modulating agent is applied to induce certain desired or selected neural pathways within the central auditory system to remain active. The plasticity modulating agent is thus used to reinforce pathways that are going to relay impulses configured to evoke a hearing sensation by the brain. This may enhance the effectiveness of the electrical stimulation signals representing a sound signal, and thereby lead to improved speech coding strategies

In certain embodiments, the ability to modulate the neural plasticity may provide a clinician or other user with the ability to tune the response of the central auditory system to electrical stimulation signals representing a sound signal. Specifically, embodiments of the present invention may provide a clinician with the ability to use a plasticity modulating agent to train the central auditory system to respond in a desired or predicted manner to the application of stimulation signals representing a sound signal.

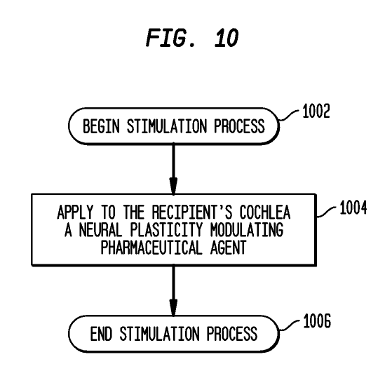

FIG. 10 is a high level flowchart illustrating the operations performed by a cochlear implant in accordance with the above embodiments of the present invention. The process begins at block 1002. At block 1004, a neural plasticity modulating agent is applied to one or more regions of the recipient's nerve cells to influence and/or control the neural plasticity of the system. As explained above, the plasticity modulating agent may be applied in a spatial and temporal manner so that a desired effect on the central auditory system is obtained. The process then ends at block 1008.

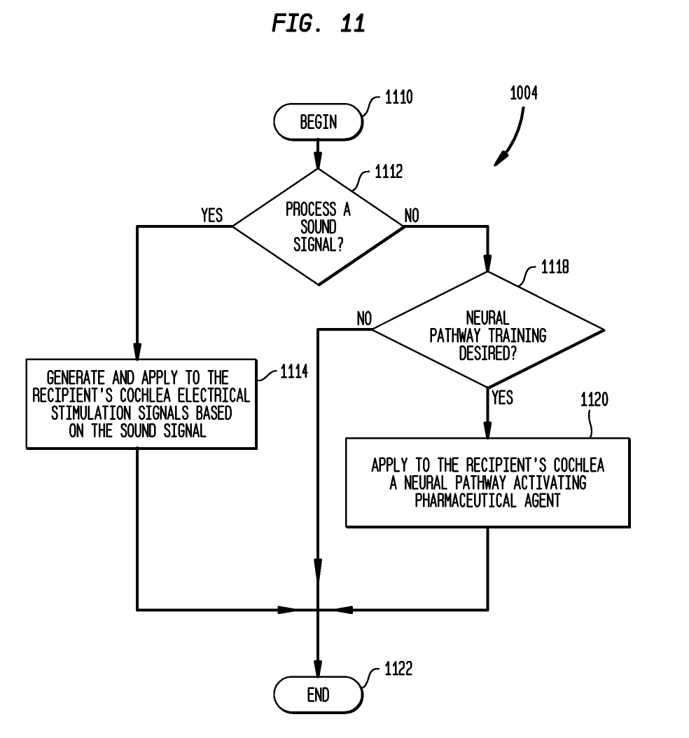

FIG. 11 is a detailed flowchart illustrating the operations that may be performed in accordance with specific embodiments of block 1004 of FIG. 10. The operations begin at block 1110. At block 1112, a decision is made if a sound signal has been received and/or whether the signal should be processed. If a received sound signal is to be processed, the method progresses to block 1114. At block 1114, electrical stimulation signals based on the received sound signal are generated and applied to cochlea nerve cells. The operations then end at block 1122.

Returning to block 1112, if no sound signal is to be processed, the method progresses to block 1118. A block 1118 a determination is made as to whether neural pathway training is desired. In other words, a decision is made as to whether plasticity modulation which activates or induces certain pathways to remain active is desired. If neural pathway training is not desired, the method ends at block 1122. However, if neural pathway training is desired, the method continues to block 1120. At block 1120, a plasticity modulating agent is applied to the recipient's cochlea nerve cells to influence and/or control the neural plasticity of the central auditory system. The method then ends at block 1122.

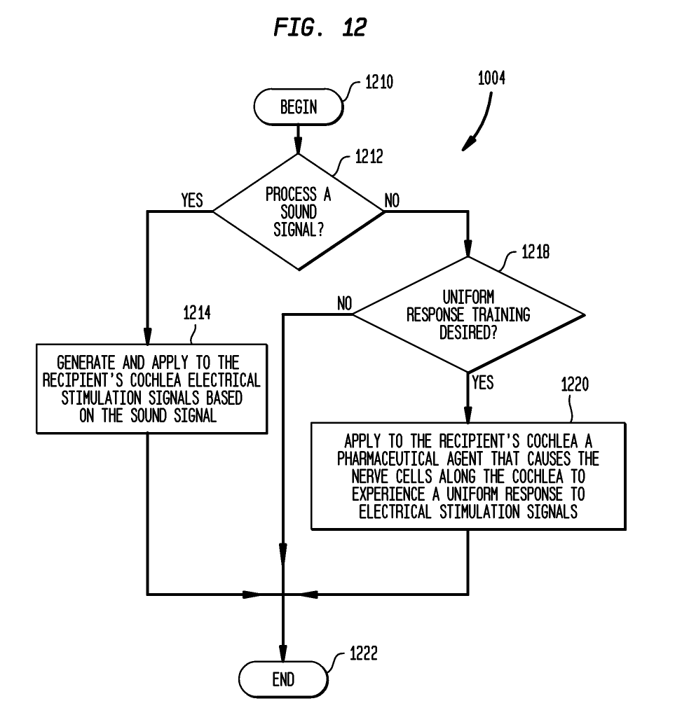

As described above, in embodiments of the present invention a plasticity modulating agent is applied to the recipient's cochlea in order to provide a substantially uniform response of the cochlea to electrical stimulation signals. Specifically, a plasticity modulating agent is applied to cochlear nerve cells at one or more locations along the cochlea. The plasticity modulating agent adjusts or modifies the response of those nerve cells to electrical stimulation to match the response of other nerve cells located at a different region of the cochlea. Therefore, the same electrical stimulation applied to different cochlea regions will have the same effect on the stimulated nerve cells. This ability to standardize the cochlea's response to electrical stimulation signals may provide for enhanced stimulation strategies. FIG. 12 is a detailed flowchart illustrating the operations that may be performed at block 1004 of FIG. 10 in accordance with such embodiments of the present invention. The operations begin at block 1210. At block 1212, a decision is made if a sound signal has been received and/or whether the signal should be processed. If a received sound signal is to be processed, the method progresses to block 1214. At block 1214, electrical stimulation signals based on the received sound signal are generated and applied to cochlea nerve cells. The operations then end at block 1222.

Returning to block 1212, if no sound signal is to be processed, the method progresses to block 1218. A block 1218 a determination is made as to whether uniform cochlea response training is desired. In other words, a decision is made as to whether plasticity modulation which causes a standardized response of the cochlea to electrical stimulation signals is desired. If uniform response training is not desired, the method ends at block 1222. However, if uniform response training is desired, the method continues to block 1220. At block 1220, a plasticity modulating agent is applied to the recipient's cochlea nerve cells to influence and/or control the neural plasticity of one or more regions of cochlear nerve cells to standardize the response of the cochlea to electrical stimulation signals. For example, the plasticity modulating agent may be applied to the basal region of the cochlea so that the response of the basal region is modulated to match the response of the apical region. In other embodiments, the response of the apical region is modulated. The method then ends at block 1222.

As described above, in other embodiments of the present invention, the application of a plasticity modulating agent may occur concurrently with the application of electrical stimulation signals configured to evoke a hearing percept of the received sound signal.

Although embodiments of the present invention have been primarily described with reference to a cochlear implant, it should be appreciated that alternative embodiments may be implanted in a variety of stimulating medical devices or prosthetic hearing devices such as acoustic hearing aids, middle ear implants, brain stem implants, or any combination of these, or other implanted devices. For example, embodiments may be implemented in a device implantable in the cochlear nucleus, the superior olive, the nucleus of the lateral lemniscus, the inferior colliculus, the medial geniculate body, the auditory cortex, Subthalamic Nucleus (STN), the Globus Pallidus (GPi), the Thalamus, and/or any other part of the central auditory system.

It should also be appreciated that embodiments of the present invention are not limited to devices configured to stimulate a recipient's auditory system, and embodiments may be used to treat other conditions caused by the lack of natural functionality or abnormal function. For example, spinal cord injury, visual impairment, sensorineural and motorneural abnormalities, such as depression, Parkinson's disease, Alzheimer's disease may also be treated in accordance with embodiments of the present invention.

To treat problems with the visual system, plasticity modulating agents may be delivered to the retina or visual cortex, in patients suffering from loss of vision. In this regard, retinal and visual cortex implants are the two most common devices which may be used to apply such stimulation to the visually impaired. For the treatment of spinal cord injured patients, plasticity modulating agents may be delivered to various locations along the patient's spinal cord.

Furthermore, while the above description has primarily described the use of a cochlear implant to apply plasticity modulating agents, such signals could be delivered using a device that is implanted in conjunction with, or instead of a cochlear implant. Still further, an apparatus could be installed to apply plasticity modulating agents to the cochlea of the patient that does not need a cochlear implant. For example, delivery of plasticity modulating agents may be performed in conjunction with use of a middle ear implant or a hearing aid.

All documents, patents, journal articles and other materials cited in the present application are hereby incorporated by reference.

Although the present invention has been fully described in conjunction with several embodiments thereof with reference to the accompanying drawings, it is to be understood that various changes and modifications may be apparent to those skilled in the art. Such changes and modifications are to be understood as included within the scope of the present invention as defined by the appended claims, unless they depart there from.

What is claimed is:

1. A cochlear implant, comprising:
a stimulating assembly implantable in a cochlea of a recipient having at least one agent delivery port and a plurality of electrical contacts;
an electrical stimulation controller configured to generate electrical stimulation signals for application to a first population of cochlea nerve cells via one or more of the plurality of electrical contacts;
a pharmaceutical agent source configured to provide a plasticity modulating agent to the at least one delivery port for application to a second population of cochlea nerve cells; and
a pharmaceutical agent controller configured to control one or more of the pharmaceutical agent source and the at least one delivery port to cause selective application of the plasticity modulating agent to the second population of nerve cells to at least one of reinforce and activate one or more neural pathways within the recipient's central auditory system.

2. The cochlear implant of claim 1, further comprising:
a sound input component configured to receive a sound signal; and
a sound processor configured to generate data signals based on the received sound signal;
wherein the electrical stimulation controller is configured to generate the electrical stimulation signals based on the data signals, and wherein application of the electrical stimulation signals to the first population of cochlea nerve cells evokes a hearing percept by the recipient.

3. The cochlear implant of claim 1, wherein the first population of nerve cells and the second population of nerve cells comprise substantially the same nerve cells.

4. The cochlear implant of claim 1, wherein the plasticity modulating agent and the electrical stimulation signals are applied concurrently to the recipient.

5. The cochlear implant of claim 1, wherein the plasticity modulating agent comprises a naturally occurring neurotrophic agent.

6. The cochlear implant of claim 1, wherein the pharmaceutical agent controller is configured receive an external control input, and wherein the controller is configured to adjust one or more of the location and timing of the application of the plasticity modulating agent based on the control input.

7. The cochlear implant of claim 1, wherein the pharmaceutical agent controller is configured to adjust one or more of the location and timing of the application of the plasticity modulating agent based on a measured neural response.

8. The cochlear implant of claim 1, wherein the implant is configured to operate in a plurality of modes of operation, and wherein the pharmaceutical agent controller is configured to apply the plasticity modulating agent only when the implant is in a selected mode of operation.

9. The cochlear implant of claim 1, wherein the pharmaceutical agent controller is configured to apply the plasticity modulating signals only when the electrical stimulation controller is not generating electrical stimulation signals.

10. The cochlear implant of claim 1, wherein the pharmaceutical agent source comprises a reservoir configured to be implanted underneath and proximate to the skin of the recipient.

11. The cochlear implant of claim 10, wherein the reservoir includes a post-operatively accessible refill port.

12. The cochlear implant of claim 10, wherein the stimulating assembly has a second reservoir disposed therein that is fluidically coupled via a catheter to the reservoir adjacent the recipient's skin, and coupled to the at least one delivery port via a channel.

13. The cochlear implant of claim 1, wherein the pharmaceutical agent source comprises a reservoir positioned in the stimulating assembly fluidically coupled to the at least one delivery port.

14. The cochlear implant of claim 13, wherein the reservoir is fluidically coupled to a post-operatively accessible refill element.

15. The cochlear implant of claim 1, wherein the at least one agent delivery port comprises a plurality of agent delivery ports.

16. The cochlear implant of claim 1, wherein the pharmaceutical agent source comprises an active infusion device operated under the control of the pharmaceutical agent controller.

17. The cochlear implant of claim 1, wherein the pharmaceutical agent source comprises a passive infusion device operated under the control of the pharmaceutical agent controller.

18. The cochlear implant of claim 1, wherein the pharmaceutical agent source comprises an ion pump operated under the control of the pharmaceutical agent controller.

19. The cochlear implant of claim 1, wherein the pharmaceutical agent source comprises:
a reservoir configured to store the plasticity modulating agent;
an agent chamber configured to receive an amount of the agent from the reservoir;
a first unidirectional valve disposed between the reservoir and the agent chamber, and a second unidirectional valve disposed between the agent chamber and an outlet port; and
an element under the control of the pharmaceutical agent controller configured to expand on or more of the plasticity modulating agent within the agent chamber and the agent chamber so as to cause release of a portion of the agent from the agent chamber via the second unidirectional valve.

20. The cochlear implant of claim 1, wherein the at least one delivery port comprises an electrically operable ion channel.

21. The cochlear implant of claim 1, wherein the pharmaceutical agent controller is configured to control the flow rate of the plasticity modulating agent through the at least one delivery port.

22. A method for modulating the neural plasticity of a recipient's auditory system with an implant comprising a stimulating assembly implantable proximate to the auditory system, the assembly having at least one agent delivery port and a plurality of electrical contacts, the method comprising:
generating electrical stimulation signals;
applying the electrical stimulation signals to a first population of cochlea nerve cells via one or more of the plurality of electrical contacts; and
applying a plasticity modulating agent to a second population of cochlea nerve cells via the at least one delivery port to at least one of reinforce and activate one or more neural pathways within the recipient's central auditory system.

23. The method of claim 22, further comprising:
receiving a sound signal at a sound input component;
generating data signals based on the received sound signal;
generating the electrical stimulation signals based on the data signals; and
applying the electrical stimulation signals to the first population of cochlea nerve cells to evoke a hearing percept by the recipient.

24. The method of claim 22, further comprising:
applying the plasticity modulating agent and the electrical stimulation signals to substantially the same nerve cell populations.

25. The method of claim 22, further comprising:
applying the plasticity modulating agent and the electrical stimulation signals to different nerve cell populations.

26. The method of claim 22, further comprising:
concurrently applying the plasticity modulating agent and the electrical stimulation signals.

27. The method of claim 22, further comprising:
measuring the recipient's neural response to an electrical stimulation signal; and
adjusting one or more of the location and timing of the application of the plasticity modulating agent based on a measured neural response.

28. The method of claim 22, wherein the implant is configured to operate in a plurality of modes of operation, the method further comprising:
generating the plasticity modulating agent only when the implant is in a selected mode of operation.

29. The method of claim 22, wherein the implant comprises a pharmaceutical agent source fluidically coupled to the at least one delivery port, and wherein applying the plasticity modulating agent to the second population of cochlea nerve cells comprises:
electrically controlling the release of the plasticity modulating agent from pharmaceutical agent source.

30. The method of claim 22, wherein applying the plasticity modulating agent to the second population of cochlea nerve cells comprises:
electrically controlling the release of the plasticity modulating agent from the at least one delivery port.

31. The method of claim 22, wherein the implant further comprises a pharmaceutical agent reservoir configured to be implanted underneath, and adjacent to the skin of the recipient, the method further comprising:
refilling the pharmaceutical agent reservoir via a post-operatively accessible refill port.

32. A stimulating medical device, comprising:
a stimulating assembly implantable proximate to a recipient's neural system, the assembly having at least one agent delivery port and a plurality of electrical contacts;
an electrical stimulation controller configured to generate electrical stimulation signals for application the neural system via one or more of the plurality of electrical contacts;
a pharmaceutical agent source configured to provide a plasticity modulating agent to the at least one delivery port for application to the neural system; and
a pharmaceutical agent controller configured to control one or more of the pharmaceutical agent source and the at least one delivery port to cause selective application of the plasticity modulating agent to the neural system to at least one of reinforce and activate one or more neural pathways within the system.

33. The stimulating medical device of claim 32, further comprising:
a sound input component configured to receive a sound signal; and
a sound processor configured to generate data signals based on the received sound signal;
wherein the electrical stimulation controller is configured to generate the electrical stimulation signals based on the data signals, and wherein application of the electrical stimulation signals to neural system evokes a hearing percept by the recipient.

34. The stimulating medical device of claim 32, wherein the plasticity modulating agent and the electrical stimulation signals are applied concurrently to the neural system.

35. The stimulating medical device of claim 32, wherein the plasticity modulating agent comprises a naturally occurring neurotrophic agent.

36. The stimulating medical device of claim 32, wherein the pharmaceutical agent controller is configured receive an external control input, and wherein the controller is configured to adjust one or more of the location and timing of the application of the plasticity modulating agent based on the control input.

37. The stimulating medical device of claim 32, wherein the pharmaceutical agent controller is configured to adjust one or more of the location and timing of the application of the plasticity modulating agent based on a measured neural response.

38. The stimulating medical device of claim 32, wherein the device is configured to operate in a plurality of modes of operation, and wherein the pharmaceutical agent controller is configured to apply the plasticity modulating agent only when the device is in a selected mode of operation.

39. The stimulating medical device of claim 32, wherein the pharmaceutical agent controller is configured to apply the plasticity modulating signals only when the electrical stimulation controller is not generating electrical stimulation signals.

40. The stimulating medical device of claim 32, wherein the pharmaceutical agent source comprises a reservoir configured to be implanted underneath and proximate to the skin of the recipient.

41. The stimulating medical device of claim 10, wherein the reservoir includes a post-operatively accessible refill port.

42. The stimulating medical device of claim 10, wherein the stimulating assembly has a second reservoir disposed therein that is fluidically coupled via a catheter to the reservoir adjacent the recipient's skin, and coupled to the at least one delivery port via a channel.

43. The stimulating medical device of claim 32, wherein the at least one agent delivery port comprises a plurality of agent delivery ports.

44. The stimulating medical device of claim 32, wherein the pharmaceutical agent source comprises an active infusion device operated under the control of the pharmaceutical agent controller.

45. The stimulating medical device of claim 32, wherein the pharmaceutical agent source comprises a passive infusion device operated under the control of the pharmaceutical agent controller.

46. The stimulating medical device of claim 32, wherein the pharmaceutical agent source comprises an ion pump operated under the control of the pharmaceutical agent controller.

47. The stimulating medical device of claim 32, wherein the pharmaceutical agent source comprises:
a reservoir configured to store the plasticity modulating agent;
an agent chamber configured to receive an amount of the agent from the reservoir;
a first unidirectional valve disposed between the reservoir and the agent chamber, and a second unidirectional valve disposed between the agent chamber and an outlet port; and
an element under the control of the pharmaceutical agent controller configured to expand on or more of the plasticity modulating agent within the agent chamber and the agent chamber so as to cause release of a portion of the agent from the agent chamber via the second unidirectional valve.

48. The stimulating medical device of claim 32, wherein the at least one delivery port comprises an electrically operable ion channel.

49. The stimulating medical device of claim 32, wherein the pharmaceutical agent controller is configured to control the flow rate of the plasticity modulating agent through the at least one delivery port.
